(19) United States
(12) Patent Application Publication
(10) Pub. No.: US 2006/0013792 A1
(43) Pub. Date: Jan. 19, 2006

(54) Solid water-in-oil cosmetic emulsion

(76) Inventors: Jacqueline Fontaine, la Celle Saint Cloud (FR); Agnes Themens, Bourg la Reine (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/182,801

(22) Filed: Jul. 18, 2005

Related U.S. Application Data
(60) Provisional application No. 60/601,989, filed on Aug. 17, 2004.

(30) Foreign Application Priority Data
Jul. 16, 2004 (FR) 04 51549

(51) Int. Cl. A61K 8/89

(52) U.S. Cl. 424/70.12

(57) ABSTRACT

Disclosed is a solid water-in-oil emulsion comprising an aqueous phase emulsified with at least one emulsifying surfactant in a fatty phase comprising at least one ester oil and at least one wax, wherein the at least one ester oil is present in an amount greater than the at least one wax and wherein the emulsion has, after at least one month at 37° C., a hardness wherein the penetration force is greater than or equal to 45 g. Also disclosed is a non-therapeutic cosmetic process for making up or caring for the skin, comprising the application to the skin of a composition comprising said emulsion.

This application claims benefit of U.S. Provisional Application No. 60/601,989, filed Aug. 17, 2004, the contents of which are incorporated herein by reference. This application also claims benefit of priority under 35 U.S.C. § 119 to French Patent Application No. 04 51549, filed Jul. 16, 2004, the contents of which are also incorporated by reference.

The present disclosure relates to a solid water-in-oil emulsion comprising at least one wax and at least one oil, which may be used in the cosmetics field. The disclosure also relates to a process for making up or caring for human skin, comprising the application of a composition comprising the solid water-in-oil emulsion to the skin.

The makeup composition according to the disclosure may be a skin makeup composition, such as a foundation, an eyeshadow, a makeup rouge, a concealer product, a body makeup product or a lipstick. For example, the makeup composition may be a foundation composition.

The care composition may be, for example, a skincare product such as a skincare base, a care cream (day cream, night cream or anti-wrinkle cream), a makeup base; a lipcare composition (lip balm); an antisun composition or self-tanning composition; or a deodorant.

Skin makeup products, for example, foundations are known in very diverse galenical forms, for example, loose powders, compact powders, cast solid products, sticks, and fluid creams.

The cast solid products may be, for example, anhydrous or in the form of emulsions. These emulsions may, for example, comprise fatty substances such as oils and solid waxes, water and a particulate phase composed, for example, of fillers and pigments.

A solid emulsion generally refers to an emulsion that does not flow under its own weight at room temperature and which may be suitable for packaging in a case: to apply the product, the user can take the product up directly by crumbling it using the fingers or an applicator such as a sponge. For good uptake of the product by the user, it may be necessary for the solid emulsion to have a satisfactory hardness and for this hardness to vary little over time, for example, after storage at temperatures above room temperature (25° C.). However, certain solid emulsions may show a drop in their hardness over time: e.g., the product can have a tendency to soften, making its uptake more difficult, or even unpleasant for the user, who has difficulty in controlling the amount of product removed during the uptake. Furthermore, an initially solid product that becomes soft is not appealing to the user.

Disclosed herein is a solid makeup or skincare composition that has a stable hardness over time, for example, after storage for at least one month at 37° C.

Disclosed herein is a solid composition that has good hardness while at the same time having a rich, creamy texture when applied to the skin.

The inventors have discovered that by using an ester oil, a wax, an emulsifying surfactant and in other embodiments, additional particular ingredients, it is possible to obtain a solid emulsion that has stable hardness over time, for example, after one month at 37° C., and that also has a rich, creamy texture when applied to the skin.

One embodiment of the disclosure is a solid water-in-oil emulsion comprising an aqueous phase emulsified with at least one emulsifying surfactant in a fatty phase comprising at least one ester oil and at least one wax, wherein the at least one ester oil is present in an amount greater than the at least one wax and wherein the emulsion has, after at least one month at 37° C., a hardness wherein the penetration force is greater than or equal to 45 g.

In another embodiment, the solid water-in-oil emulsion comprises at least two polyols.

In a further embodiment, the solid water-in-oil emulsion comprises at least one pasty fatty substance.

In yet another embodiment, the solid water-in-oil emulsion comprises at least one hydrophobic coated pulverulent material in an amount of less than or equal to 20% by weight with respect to the total weight of the composition.

Another embodiment of the disclosure is a non-therapeutic cosmetic process for making up or caring for the skin, comprising the application to the skin of a solid water-in-oil composition as defined above.

As used herein, the term “solid composition” means a composition that does not flow under its own weight at room temperature (25° C.) after one hour.

As used herein, the term “ester oil” means an oil that is liquid at room temperature (25° C.) comprising at least one ester function in its molecule. The ester oil may be, for example, a monoester.

The ester oil may, for example, be chosen from the monoesters of formula R₁COOR₂ wherein:
R₁ is chosen from linear and branched hydrocarbon-based chains comprising from 4 to 40 carbon atoms, for example, from 4 to 30 carbon atoms and further, for example, from 7 to 20 carbon atoms, and
R₂ is chosen from branched hydrocarbon-based chains comprising from 3 to 40 carbon atoms, for example, from 10 to 30 carbon atoms and further, for example, from 16 to 26 carbon atoms.

Non-limiting examples of ester oils that may be mentioned include isodecyl neopentanoate; isocetyl octanoate; isononyl isononanoate, isodecyl isononanoate, tridecyl isononanoate; hexyl laurate, 2-hexyldecyl laurate; isopropyl myristate, isocetyl myristate, isotridecyl myristate, 2-octyldodecyl myristate; isopropyl palmitate, 2-ethylhexyl palmitate, isooctyl palmitate, isocetyl palmitate, isodecyl palmitate, isostearyl palmitate, 2-octyldecyl palmitate; isopropyl isostearate, 2-octyldodecyl stearate, isostearyl isostearate; 2-octyldodecyl erucate; and mixtures thereof.

The at least one ester oil may be present in the emulsion according to the disclosure in an amount ranging from 5% to 20% by weight, for example, ranging from 7% to 15% by weight and, further, for example, ranging from 8% to 12% by weight, relative to the total weight of the composition.

In one embodiment, the fatty phase may also comprise at least one volatile silicone oil.

As used herein, the term “volatile oil” means an oil (or non-aqueous medium) that can evaporate on contact with the skin in less than one hour, at room temperature and atmospheric pressure. The volatile oil may be a volatile cosmetic oil, which is liquid at room temperature, for example, having a non-zero vapour pressure at room temperature and atmospheric pressure, for example, having a vapour pressure ranging from 0.13 Pa to 40 000 Pa (10⁻³ to 300 mmHg), for example, ranging from 1.3 Pa to 13 000 Pa (0.01 to 100 mmHg) and further, for example, ranging from 1.3 Pa to 1300 Pa (0.01 to 10 mmHg).

The volatile silicone oil may be chosen from linear or cyclic silicone oils with a viscosity at room temperature of less than 8 mm²/s, for example, oils comprising from 2 to 7 silicon atoms, these silicones optionally comprising alkyl or alkoxy groups comprising from 1 to 10 carbon atoms. As volatile silicone oils that may be used in the disclosure, non-limiting mention may be made of octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

The volatile silicone oil may be present in an amount ranging from 5% to 40% by weight, for example, ranging from 10% to 30% by weight and, further, for example, ranging from 15% to 30% by weight, relative to the total weight of the composition.

In one embodiment, the total content of water and of volatile silicone oil in the composition is less than or equal to 50% by weight, for example, from 30% to 50% by weight and further, for example, from 40% to 50% by weight, relative to the total weight of the composition.

In an embodiment, the fatty phase of the emulsion may comprise at least one additional oil other than the ester oils and the volatile silicone oils described above.

The at least one additional oil may be chosen, for example, from volatile or non-volatile hydrocarbon-based oils and fluoro oils.

As used herein, the term “hydrocarbon-based oil” means an oil comprising carbon and hydrogen atoms, and possibly oxygen and nitrogen atoms, and comprising no silicon or fluorine atoms. It may comprise alcohol, ether, carboxylic acid, amine and/or amide groups.

As used herein, the term “fluoro oil” means an oil comprising at least one fluorine atom.

As the at least one additional volatile oil that may be used in the disclosure, non-limiting mention may be made of:
hydrocarbon-based volatile oils comprising from 8 to 16 carbon atoms, and mixtures thereof, for example, branched C₈-C₁₆ alkanes, for instance C₈-C₁₆ isoalkanes (also known as isoparaffins), isododecane, isodecane and isohexadecane, for instance the oils sold under the trade names “Isopar” and “Permethyl”, branched C₈-C₁₆ esters, for instance isohexyl neopentanoate and mixtures thereof; and
volatile fluoro oils such as nonafluoroethoxybutane, nonafluoromethoxybutane, decafluoropentane, tetradecafluorohexane and dodecafluoropentane.

As the at least one additional non-volatile oil that may be used in the disclosure, non-limiting mention may be made of hydrocarbon-based oils of mineral or synthetic origin, such as linear or branched hydrocarbons, for instance liquid paraffin or derivatives thereof, liquid petroleum jelly, polydecenes, hydrogenated polyisobutene such as Parleam sold by the company Nippon Oil Fats, and squalane of synthetic or plant origin.

For example, the at least one additional non-volatile oil may also be chosen from
hydrocarbon-based oils of plant origin with a high triglyceride content, comprising fatty acid esters of glycerol, the fatty acids of which may have varied chain lengths, these chains possibly are linear or branched, and saturated or unsaturated, for example, triglycerides of a fatty acid, for example, comprising from 4 to 22 carbon atoms, for instance heptanoic or octanoic acid triglycerides, and capric/caprylic acid triglycerides, or alternatively hydroxylated triglycerides, such as sweet almond oil, beauty-leaf oil, palm oil, grapeseed oil, sesame seed oil, arara oil, rapeseed oil, sunflower oil, cottonseed oil, apricot oil, castor oil, alfalfa oil, marrow oil, blackcurrant oil, macadamia oil, musk rose oil, hazelnut oil, avocado oil, jojoba oil, olive oil, cereal (corn, wheat, barley or rye) germ oil and shea butter oil;
C₈-C₂₆ higher fatty acids such as oleic acid, linoleic acid, linolenic acid or isostearic acid; and
C₈-C₂₆ higher fatty alcohols such as oleyl alcohol, linoleyl alcohol, linolenyl alcohol, isostearyl alcohol or octyldodecanol; synthetic ethers comprising at least 7 carbon atoms, silicone oils such as linear, optionally phenylated polydimethylsiloxanes (PDMS) that are liquid at room temperature, such as phenyl trimethicones, phenyl trimethylsiloxy diphenylsiloxanes, diphenyl dimethicones, diphenyl methyldiphenyl trisiloxanes and 2-phenylethyl trimethylsiloxysilicates, which are liquid, optionally substituted with aliphatic and/or aromatic groups, for instance alkyl, alkoxy or phenyl groups, which are pendent and/or at the end of a silicone chain, these groups comprising from 2 to 24 carbon atoms and are optionally fluorinated, or with functional groups such as hydroxyl, thiol and/or amine groups.

For example, the at least one additional oil may be present in an amount ranging from 0.0% to 10% by weight, for example, ranging from 0.1% to 10% by weight, such as from 0.1 to 5% by weight relative to the total weight of the composition. In one embodiment, the content of additional oil is 0% by weight, relative to the total weight of the composition.

According to one embodiment of the disclosure, the emulsion comprises as the sole oils, an ester oil, and optionally at least one volatile silicone oil, as described above.

In one embodiment, the at least one wax present in the emulsion according to the disclosure may be chosen from waxes with a hardness ranging from 5 MPa to 9 MPa, for example, ranging from 6 MPa to 9 MPa and further, for example, ranging from 7 MPa to 9 MPa.

As used herein, the term “wax” means a lipophilic fatty compound that is solid at room temperature (25° C.), with a reversible solid/liquid change of state, having a melting point of greater than 30° C. which may be up to 200° C., a hardness of greater than 0.5 MPa, and having an anisotropic crystal organization in the solid state. By bringing the wax to its melting point, it is possible to make it miscible with oils and to form a microscopically homogeneous mixture, but on returning the temperature of the mixture to room temperature, recrystallization of the wax in the oils of the mixture is obtained.

The hardness of the wax may be determined by measuring the compression force, at 20° C. using the texturometer sold under the name TA-XT2i by the company Rheo, equipped with a stainless-steel cylinder, 2 mm in diameter, travelling at a measuring speed of 0.1 mm/s, and penetrating into the wax to a penetration depth of 0.3 mm. To perform the hardness measurement, the wax is melted at a temperature equal to the melting point of the wax+20° C. The molten wax is poured into a container 30 mm in diameter and 20 mm deep. The wax is recrystallized at room temperature (25° C.) for 24 hours and is then stored for at least 1 hour at 20° C. before performing the hardness measurement. The hardness value is the measured compression force divided by the surface area of the texturometer cylinder in contact with the wax.

For example, the at least one wax may be chosen from hydrocarbon-based waxes, silicone waxes and/or fluoro waxes, optionally comprising ester or hydroxyl functions.

The at least one wax having the hardness as defined above may be chosen from carnauba wax, microcrystalline waxes, ozokerites, hydrogenated jojoba oil, polyethylene waxes such as the wax sold under the name “Performalene 400 Polyethylene” by the company New Phase Technologies, silicone waxes, for instance poly(C₂₄-C₂₈)alkylmethyldimethylsiloxane, such as the product sold under the name “Abil Wax 9810” by the company Goldschmidt, palm butter, the C₂₀-C₄₀ alkyl stearate sold under the name “Kester Wax K82H” by the company Kester Keunen, stearyl benzoate, shellac wax, and mixtures thereof. A wax chosen from carnauba wax, candelilla wax, ozokerites, hydrogenated jojoba oil and polyethylene waxes may be used. The wax may be, for example, chosen from candelilla wax and ozokerite, and mixtures thereof.

The wax may be present in the composition according to the disclosure in an amount ranging from 1% to 10% by weight, for example, ranging from 2% to 7% by weight and, further, for example, ranging from 2% to 5% by weight, relative to the total weight of the composition.

The emulsion according to the disclosure comprises at least one emulsifying surfactant that allows a water-in-oil emulsion to be obtained, for example, a surfactant having an HLB (hydrophilic/lipophilic balance) of less than 7; an emulsifying surfactant of this kind may be, for example, chosen from fatty acid esters of polyols, for instance glyceryl or sorbitol mono-, di-, tri- or sesqui-oleates or stearates, glyceryl or polyethylene glycol laurates; alkyl or alkoxy dimethicone copolyols comprising an alkyl or alkoxy chain that is pendent or at the end of the silicone skeleton, for example comprising from 6 to 22 carbon atoms; and polymers such as polyoxyalkylenated fatty acid esters of glycol. For example, use may also be made of emulsifying silicone elastomers and polyhydroxylated silicones, for example, polyglycerolated silicones. It is possible, for example, to use a mixture of the aforementioned surfactants.

For example, the emulsifying surfactant may be a C₈-C₂₂ alkyl dimethicone copolyol, for example, an oxypropylenated and/or oxyethylenated polymethyl-(C₈-C₂₂)alkyldimethylmethylsiloxane.

The C₈-C₂₂ alkyl dimethicone copolyol may be chosen from compounds of Formula (I): 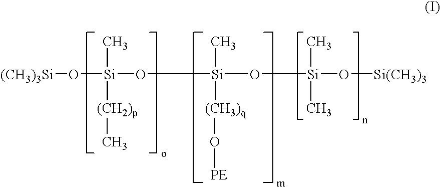
wherein:
PE is (—C₂H₄O)ₓ—(C₃H₆O)_{y}—R,
wherein:
R is chosen from a hydrogen atom and alkyl radicals comprising from 1 to 4 carbon atoms,
x is a number ranging from 0 to 100, and
y is a number ranging from 0 to 80, wherein x and y are not simultaneously equal to 0,
m is a number ranging from 1 to 40,
n is a number ranging from 10 to 200,
o is a number ranging from 1 to 100,
p is a number ranging from 7 to 21, and
q is a number ranging from 0 to 4.
   According to one embodiment, for example:
R is H
m is a number ranging from 1 to 10
n is a number ranging from 10 to 100
o is a number ranging from 1 to 30
p is a number ranging from 15 and
q is a number ranging from 3.

As a C₈-C₂₂ alkyl dimethicone copolyol non-limiting mention may be made of cetyl dimethicone copolyol, such as the product sold under the name Abil EM-90 by the company Goldschmidt.

The emulsifier may also be, for example, a silicone surfactant having a polyhydroxylated chain, for example, a polyglycerolated chain. As used herein, “polyhydroxylated chain” means a hydrocarbon chain comprising at least two hydroxyl groups.

For example, polyhydroxylated silicones may be chosen from formula (I′):
R¹ₐR²_{b}R³_{c}SiO_{(4-a-b-c)/2} (I′)
wherein:
a) a is a number ranging from 1 to 2.5; and
   b and c, which may be identical or different, are each chosen from a number ranging from 0.001 to 1.5,
b) R¹, which may be identical or different, is chosen from:
   C₁ to C₃₀ alkyl radicals, optionally substituted with at least one fluorine atoms and amino and/or carboxyl groups,
   aryl and aralkyl radicals, and
   the radicals of general formula (II):
      C_{d}H_{2d}—O—(C₂H₄O)ₑ(C₃H₆O)_{f}R⁴ (II)
   wherein:
      R⁴ is chosen from C₄ to C₃₀ hydrocarbon-based radicals or radical R⁵—(CO)— wherein R is chosen from a C₁ to C₃₀ hydrocarbon-based radical, and
      d is a number ranging from 0 to 15, and
      e and f, which may be identical or different, are each numbers ranging from 0 to 50, and
   combinations thereof,
c) R² is chosen from compounds of formula (III):
   -Q-O—X (III)
   wherein:
      Q is chosen from divalent C₂ to C₂₀ hydrocarbon-based radicals optionally comprising at least one ether bond and/or at least one ester bond, and
      X is a polyhydroxylated hydrocarbon-based radical,
d) R³ is chosen from an organosiloxane group of formula (IV): 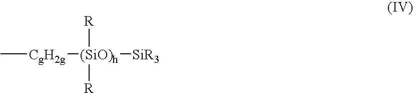
   wherein:
   R, which may be identical or different, is each chosen from C₁ to C₃₀ alkyl radicals optionally substituted with at least one fluorine atoms; aryl radicals; aralkyl radicals; and radical R¹ as defined above.
      g is a number ranging from 1 to 5 and
      h is a number ranging from 0 to 500.

It should be noted that the radicals R¹, R² and R³ of the silicone polymers of general formula (I′), as defined above, are not sequentially distributed, but are randomly distributed, i.e. they appear in the structure of the polymer without any determined order.

For example, in a) above:
a, for example, may range from 1.2 to 2.3 and, b and c, which may be idenitcal or different, may, for example, range from 0.05 to 1.

For example, in b) above:
when R¹ is chosen from alkyl radicals, for example, C₁ to C₃₀ alkyl radicals, further, for example, C₁ to C₂₅ alkyl radicals, and even further, for example, C₁ to C₂₀ alkyl radicals, even further, for example, C₁ to C₁₀ alkyl radicals and even further, for example, C₁ to C₆ alkyl radicals, and even further, for example, C₁ to C₄ alkyl radicals. For example, R¹ may be chosen from methyl, ethyl, n-propyl, isopropyl, n-butyl, isobutyl, tert-butyl, pentyl, hexyl, heptyl, octyl, nonyl, decyl, undecyl or lauryl radicals. R¹ may also be chosen from cycloalkyl radicals such as a cyclopropyl, a cyclobutyl, a cyclopentyl or a cyclohexyl. R¹ may also be chosen from linear or branched monounsaturated or polyunsaturated alkyl radicals. R¹ may also be chosen from alkyl radicals substituted with at least one fluorine atoms, such as trifluoropropyl or heptadecafluorodecyl. R¹ may also be chosen from alkyl radicals substituted with at least one amino groups, such as 2-aminoethyl, 3-aminopropyl or 3-(2-aminoethyl)aminopropyl. R¹ may also be chosen from alkyl groups substituted with at least one carboxyl groups, such as 3-carboxypropyl;
R¹ may also be chosen from aryl radicals and aralkyl radicals such as phenyl radicals, tolyl radicals, benzyl radicals or phenethyl radicals.

According to one embodiment, R¹ may be chosen from hydroxylated radicals and radicals derived from the addition reaction of a saturated or unsaturated, linear or branched alkenyl ether, wherein d=0 and thus of formula:
—O—(C₂H₄O)ₑ(C₃H₆O)_{f}R⁴

According to one embodiment, when e and f are equal to zero, then R¹ is chosen from alkoxy groups comprising from 4 to 30 carbon atoms, for example a C₄ to C₁₀ lower alkoxy radical, such as butoxy or pentoxy, or a C₁₁ to C₃₀ higher alkoxy radical, such as oleoxy or stearoxy, for example, cetyl alcohol, oleyl alcohol and stearyl alcohol, or a radical derived from an acid or from a fatty acid, such as acetic acid, lactic acid, butyric acid, oleic acid, stearic acid or behenic acid.

According to another embodiment, when e and f are greater than 1, then R¹ is a hydroxyl radical derived from the addition reaction of an alkylene oxide.

According to yet another embodiment, when e and f are equal to zero, d may be equal to 3, 5 or 11. In this case, R¹, depending on the nature of the substituent R⁴, is an allyl ether, pentenyl ether or undecenyl ether radical, or an allyl stearyl ether, pentenyl behenyl ether or undecenyl oleyl ether radical.

When e and f are other than zero, an alkoxy radical and an ester radical may be present via a polyoxyalkylene group.

In one embodiment, d is a number ranging from 3 to 5.

According to one embodiment, the radical R¹ may be any one of the radicals defined above, or a combination of two or more of these radicals.

For example, R¹ is an alkyl radical chosen from a methyl radical and a lauryl radical, and combinations thereof.

When R¹ corresponds to two or more radicals, for example a methyl radical and a lauryl radical, these radicals appear randomly in the structure, and with a frequency that may be different for each of the radicals.

In one embodiment, at least 50% of the radicals R¹, for example, at least 70% of the radicals R¹ and further, for example, 100% of the radicals R¹, are methyl radicals.

For example, in c) above:
Q may be chosen from divalent hydrocarbon-based radicals chosen from:
—(CH₂)₂—, —(CH₂)₃—, —CH₂CH(CH₃)—CH₂, —(CH₂)₄—, —(CH₂)₅—, —(CH₂)₆—, —(CH₂)₇—, —(CH₂)₈—, —(CH₂)₂—CH(CH₂CH₂—CH₃)—, —CH₂—CH(CH₂CH₃)—, —(CH₂)₃—O—(CH₂)₂—, —(CH₂)₃—O—(CH₂)₂—O—(CH₂)₂—, —(CH₂)₃—O—CH₂CH(CH₃)— and —CH₂—CH(CH₃)—COO(CH₂)₂—.

For example, Q may be a divalent radical chosen from —(CH₂)₂— and —(CH₂)₃—.
X may be, for example, a polyhydroxylated hydrocarbon-based radical comprising at least two hydroxyl residues, for example, a hydrocarbon-based group chosen from glyceryl derivatives and saccharide derivatives.

The glycerol residues may be compounds chosen from 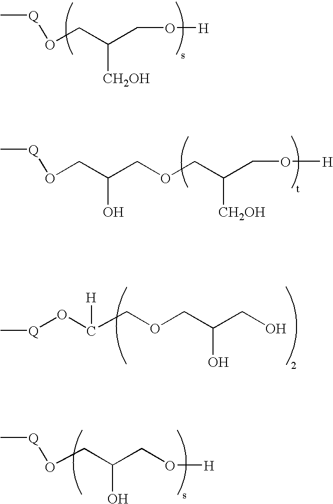
wherein:
Q has the same meaning as in formula (III) above, and
s and t, which may be identical or different, are chosen from numbers ranging from 1 to 20, for example, from 1 to 15, further, for example, from 1 to 10 and even further, for example, from 1 to 5.

In the above formulae, at least one hydroxyl group may be replaced with one or more alkoxy groups or ester groups.

The saccharide radicals that may be used in formula (III) may be chosen from the monosaccharide type, such as glycosyl, mannosyl, galactosyl, ribosyl, arabinosyl, xylosyl or fructosyl groups, the oligosaccharide type, such as maltosyl, cellobiosyl, lactosyl or maltotriosyl, and the polysaccharide type, such as cellulose or starch.

For example, the saccharide groups may be chosen from the monosaccharide and oligosaccharide types.

For example, in d) above:
R, which may be identical or different, may be chosen from C₁ to C₂₀, for example, C₁ to C₁₀ and further, for example, C₁ to C₆ alkyl radicals, optionally substituted with at least one fluorine atoms. When the R is chosen from the alkyl radicals as defined above, optionally substituted with at least one fluorine atoms, they have the same meaning as the radical R¹ as defined above.
g may be equal to 2.
h may be chosen from a number ranging from 1 to 50.

According to one embodiment, the silicone polymer of general formula (I′) is one wherein:
a is a number ranging from 1 to 1.4, and b and c, which may be identical or different, are each a number ranging from 0.02 to 0.03, and
R¹ is chosen from a C₁ to C₁₀, for example, C₁ to C₆ and further, for example, C₁ to C₄, alkyl radicals,
R² is chosen from compounds of formula (IIIA):
   —C₃H₆O[CH₂CH(OH)CH₂O]ₙH (IIIA)
wherein:
n is a number ranging from 1 to 5, and
R³ is chosen from compounds of formula (IVA):
   —C₂H₄(CH₃)₂SiO[(CH₃)₂SiO]ₘSi(CH₃)₃ (IVA)
wherein:
m is a number ranging from 3 to 9.

According to another embodiment, the silicone polymer of general formula (I′), which may be used in the cosmetic compositions according to the disclosure, is one wherein:
a is a number ranging from 1 to 1.4, and b and c, which may be identical or different, are numbers ranging from 0.02 to 0.04,
R¹ is a methyl radical,
R² is chosen from compounds of formula (IIIA) wherein n is a number ranging from 1 to 5, and
R³ chosen from compounds of formula (IVA) wherein m is a number ranging from 3 to 9.

For example, the silicone polymer of general formula (I′) used in the composition according to the disclosure may be chosen from polyglyceryl-3 polymethylsiloxyethyl dimethicone, laurylpolyglyceryl-3 polymethylsiloxyethyl dimethicone and polyglyceryl-3 disiloxane dimethicone, the respective formulae of which are:
Polyglyceryl-3 polymethylsiloxyethyl dimethicone (formula (V)): 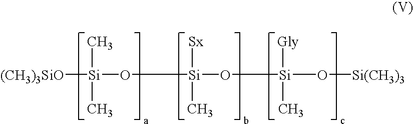
   wherein:
Sx: —C₂H₄[(CH₃)₂SiO]ₘSi(CH₃)₃
Gly: —C₃H₆O[CH₂—CH(OH)CH₂O]ₙH
and a=1-1.4, b=0.02-0.04, c=0.02-0.04, m=3-9, n=1-5
   Laurylpolyglyceryl-3 polymethylsiloxyethyl dimethicone (formula (VI)): 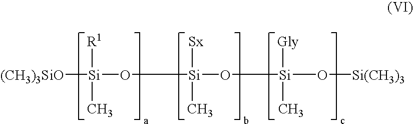
      wherein Sx, Gly, a, b, c, m and n have the same meaning as above for Polyglyceryl-3 polymethylsiloxyethyl dimethicone (formula (V)) and R¹ is chosen from methyl radicals and lauryl radicals;
   Polyglyceryl-3 disiloxane dimethicone (formula (VII)): 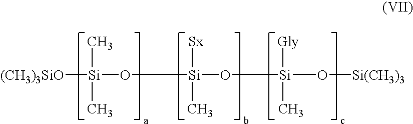
      wherein Gly, a, b, c, m and n have the same meaning as above for Polyglyceryl-3 polymethylsiloxyethyl dimethicone (formula (V)), and
      Sx: —O(CH₃)₂SiO—Si(CH₃)₃.

Silicone polymers of formula (I′) are described in detail in European Patent Application No. EP 1 213 316, which is incorporated by reference.

According to one embodiment, the silicone polymer of general formula (I′) is chosen from the polymers sold by the company Shin-Etsu under the references KF 6100, KF 6104 and KF 6105.

The polymer sold under the reference KF 6104 may be suitable for preparing the cosmetic compositions in accordance with the disclosure.

An emulsifying elastomeric crosslinked organopolysiloxane comprising, for example, a polyhydroxylated chain may also be used as an emulsifier.

The elastomeric crosslinked organopolysiloxane may be obtained by a crosslinking addition reaction of diorganopolysiloxane comprising at least one hydrogen linked to silicon and of polyhydroxylated or polyoxyalkylenated compounds comprising ethylenically unsaturated groups, optionally in the presence of a platinum catalyst.

For example, the elastomeric crosslinked organopolysiloxane may be obtained by a crosslinking addition reaction (A) of diorganopolysiloxane comprising at least two hydrogens each linked to a silicon, and (B) of glycerolated or polyoxyalkylenated compounds comprising at least two ethylenically unsaturated groups, optionally in the presence (C) of a platinum catalyst.

For example, the organopolysiloxane may be obtained by reaction of a polyglycerolated compound comprising dimethylvinylsiloxy end groups and of methylhydrogenopolysiloxane comprising trimethylsiloxy end groups, in the presence of a platinum catalyst.

Compound (A) is the base reagent for the formation of elastomeric organopolysiloxane and the crosslinking may be performed by an addition reaction of compound (A) with compound (B) in the presence of the catalyst (C).

Compound (A) may be, for example, an organopolysiloxane comprising at least 2 hydrogen atoms linked to different silicon atoms in each molecule.

Compound (A) may have any molecular structure, for example, a linear chain or branched chain structure or a cyclic structure.

Compound (A) may have a viscosity at 25° C. ranging from 1 to 50,000 centistokes, for example, to enhance miscibility with compound (B).

The organic groups linked to silicon atoms of the compound (A) may be alkyl groups comprising from 1 to 18 carbon atoms, such as methyl, ethyl, propyl, butyl, octyl, decyl, dodecyl (or lauryl), myristyl, cetyl or stearyl; substituted alkyl groups such as 2-phenylethyl, 2-phenylpropyl or 3,3,3-trifluoropropyl; aryl groups such as phenyl, tolyl or xylyl; substituted aryl groups such as phenylethyl; and substituted monovalent hydrocarbon-based groups such as an epoxy group, a carboxylate ester group or a mercapto group. The said organic group may be, for example, chosen from methyl, phenyl and lauryl groups.

Compound (A) may, for example, be chosen from methylhydrogenopolysiloxanes comprising trimethylsiloxy end groups, dimethylsiloxane-methylhydrogenosiloxane copolymers comprising trimethylsiloxy end groups, dimethylsiloxane-methylhydrogenosiloxane cyclic copolymers, or dimethylsiloxane-methylhydrogenosiloxane-laurylmethylsiloxane copolymers comprising trimethylsiloxy end groups.

Compound (B) may be chosen from:
a polyglycerolated compound corresponding to formula (B′) below:
   CₘH₂ₘ₋₁—O-[Gly]n-CₘH₂ₘ₋₁ (B′)
   wherein
m is a number ranging from 2 to 6,
n is a number ranging from 2 to 200, for example, ranging from 2 to 100, further, for example, ranging from 2 to 50, even further, for example, ranging from 2 to 20, even further, for example, ranging from 2 to 10 and even further, for example, ranging from 2 to 5, and even further, for example, equal to 3;
Gly is chosen from:
   —CH₂—CH(OH)—CH₂—O— and —CH₂—CH(CH₂OH)—O— and
a polyoxyalkylene compound having two vinyl groups.

According to one embodiment, the sum of the number of ethylenic groups per molecule of compound (B) and of the number of hydrogen atoms linked to silicon atoms per molecule of compound (A) is at least 4.

Compound (A) may, for example, be added in an amount wherein the molar ratio between the total amount of hydrogen atoms linked to silicon atoms in compound (A) and the total amount of all the ethylenically unsaturated groups in compound (B) ranges from 1/1 to 20/1.

Compound (C) is the crosslinking reaction catalyst, and may be, for example, chloroplatinic acid, chloroplatinic acid-olefin complexes, chloroplatinic acid-alkenylsiloxane complexes, chloroplatinic acid-diketone complexes, platinum black and platinum on a support.

The catalyst (C) may be, for example, added in from 0.1 to 1000 parts by weight, for example, from 1 to 100 parts by weight, as clean platinum metal per 1000 parts by weight of the total amount of compounds (A) and (B).

The emulsifying silicone elastomer may be conveyed in gel form in at least one hydrocarbon-based oil and/or one silicone oil. In these gels, the emulsifying elastomer is often in the form of non-spherical particles.

Polyoxyalkylenated elastomers are described, for example, in U.S. Pat. No. 5,236,986, U.S. Pat. No. 5,412,004, U.S. Pat. No. 5,837,793 and U.S. Pat. No. 5,811,487, the content of which is incorporated by reference.

Polyoxyalkylenated silicone elastomers that may be used include those sold under the names “KSG-21”, “KSG-20”, “KSG-30”, “KSG-31”, “KSG-32”, “KSG-33”, “KSG-210”, “KSG-310”, “KSG-320”, “KSG-330”, “KSG-340” and “X-226146” by the company Shin-Etsu and “DC9010” and “DC9011” by the company Dow Corning.

Polyglycerolated elastomers are, for example, described in PCT Patent Publication No. WO-A-2004/024798, the content of which is incorporated by reference.

Polyglycerolated silicone elastomers that may be used are those sold under the names “KSG-710”, “KSG-810”, “KSG-820”, “KSG-830” and “KSG-840” by the company Shin-Etsu.

As other surfactant that may be used in the disclosure to obtain a water-in oil emulsion, non-limiting mention may be made of polymers of the type such as polyoxyalkylenated fatty acid esters of glycol with water-in-oil emulsifying properties.

The fatty acid ester of the said polymer may be, for example, polyhydroxylated. For example, this polymer is a block polymer, for example, of ABA structure, comprising poly(hydroxylated ester blocks and polyethylene glycol blocks.

The fatty acid ester of the said emulsifying polymer defined above generally has a chain comprising from 12 to 20 carbon atoms, for example, from 14 to 18 carbon atoms. The esters may be chosen from oleates, palmitates and stearates.

The polyethylene glycol blocks of the said emulsifying polymer as defined above may comprise from 4 to 50 mols of ethylene oxide, for example, from 20 to 40 mols of ethylene oxide.

One polymer surfactant that may be, for example, suitable for producing the compositions of the disclosure is 30 EO polyethylene glycol dipolyhydroxystearate, sold under the trade name “Arlacel P 135” by the company ICI.

The emulsifying surfactant may be present in the composition in an amount ranging from 1% to 10% by weight, for example, ranging from 2% to 5% by weight, relative to the total weight of the composition.

The aqueous phase of the solid emulsion according to the disclosure comprises water. The water may be chosen from a floral water such as cornflower water; a mineral water such as eau de Vittel, eau de Lucas or eau de La Roche Posay; and a spring water. The water may be present in the emulsion according to the disclosure in an amount ranging from 10% to 40% by weight, for example, ranging from 15% to 30% by weight and further, for example, ranging from 15% to 25% by weight relative to the total weight of the composition.

The aqueous phase may also comprise at least one solvent that is water-miscible (at room temperature—25° C.), for instance monoalcohols comprising from 2 to 6 carbon atoms, such as ethanol or isopropanol;
polyols, for example, polyols comprising from 2 to 20 carbon atoms, for example, comprising from 2 to 10 carbon atoms and further, for example, comprising from 2 to 6 carbon atoms, such as glycerol, propylene glycol, butylene glycol, pentylene glycol, hexylene glycol, dipropylene glycol or diethylene glycol;
glycol ethers (for example, comprising from 3 to 16 carbon atoms) such as mono-, di- or tripropylene glycol (C₁-C₄)alkyl ethers or mono-, di- or triethylene glycol (C₁-C₄)alkyl ethers; and mixtures thereof.

According to one embodiment, the aqueous phase comprises at least one polyol which is miscible with water, for example, miscible at room temperature (25° C.). As water-miscible polyols non-limiting mention may be made of polyols having in from 3 to 20 carbon atoms, for example, having 3 to 10 carbon atoms, and further, for example, having 3 to 6 carbon atoms, such as glycerol, propylene glycol, butylene glycol, pentylene glycol, hexylene glycol, dipropylene glycol, diethylene glycol, and mixtures thereof.

The polyol or polyols may be present in an amount ranging from 5% to 20% by weight, relative to the total weight of the composition, for example, ranging from 5% to 17% by weight, further, for example, at least 6% by weight, further, for example, ranging from 6% to 20% by weight, further, for example, ranging from 6% to 17% by weight, and even further, for example, ranging from 8% to 12% by weight.

According to one embodiment, the composition may comprise at least two water-miscible polyols, for example, a water-miscible polyol having 3 carbon atoms and a water-miscible polyol having more than 3 carbon atoms, for example, having from 4 to 20 carbon atoms, further, for example, having from 4 to 10 carbon atoms and even further, for example, having from 4 to 6 carbon atoms, which can be selected from the polyols disclosed above.

The emulsion according to the disclosure may comprise at least one water-miscible organic solvent, for example, a polyol, in an amount ranging from 1% to 20% by weight, for example, ranging from 3% to 15% by weight, relative to the total weight of the composition.

The aqueous phase may also comprise at least one stabilizer chosen from, for example, sodium chloride, magnesium dichloride and magnesium sulfate.

The aqueous phase may also comprise at least one water-soluble or water-dispersible compound that is compatible with an aqueous phase chosen from, for example, gelling agents, film-forming polymers, thickeners and surfactants.

According to one embodiment, the aqueous phase may be present in the emulsion according to the disclosure in an amount ranging from 25% to 50% by weight, for example, ranging from 25% to 45% by weight and further, for example, ranging from 25% to 35% by weight, relative to the total weight of the emulsion.

The fatty phase of the emulsion may also comprise at least one pasty fatty substance.

As used herein, the term “pasty” means a lipophilic fatty compound which undergoes a reversible solid/liquid change of state, having in the solid state an anisotropic crystal organization, and comprising at a temperature of 23° C. a liquid fraction and a solid fraction.

As used herein, the term “pasty compound” means a compound having a hardness at 20° C. ranging from 0.001 to 0.5 MPa, for example, from 0.002 to 0.4 MPa.

Hardness is measured according to a method of penetration of a probe in a sample of compound, for example, using a texture analyzer (for example the TA-XT2i machine from Rheo) equipped with a stainless-steel cylinder 2 mm in diameter. The hardness measurement is performed at 20° C. at the center of five samples. The cylinder is introduced into each sample at a pre-speed of 1 mm/s and then at a measuring speed of 0.1 mm/s, the penetration depth is 0.3 mm. The hardness value revealed is that of the maximum peak.

This pasty compound is also, at a temperature of 23° C., in the form of a liquid fraction and a solid fraction. In other words, the starting melting point of the pasty compound is less than 23° C. The liquid fraction of the pasty compound measured at 23° C. is present in an amount ranging from 9% to 97% by weight of the compound. This liquid fraction at 23° C. may be present in an amount ranging from 15% to 85%, for example, from 40% to 85% by weight.

The liquid fraction by weight of the pasty compound at 23° C. is equal to the ratio of the heat of fusion consumed at 23° C. to the heat of fusion of the pasty compound.

The heat of fusion of the pasty compound is the heat consumed by the compound to change from the solid state to the liquid state. The pasty compound is said to be in the solid state when all of its mass is in solid crystalline form. The pasty compound is said to be in the liquid state when all of its mass is in liquid form.

The heat of fusion of the pasty compound is equal to the area under the curve of the thermogram obtained using a differential scanning calorimeter (DSC), such as the calorimeter sold under the name MDSC 2920 by the company TA Instrument, with a temperature rise of 5 or 10° C. per minute, according to standard ISO 11357-3:1999. The heat of fusion of the pasty compound is the amount of energy required to make the compound change from the solid state to the liquid state. It is expressed in J/g.

The heat of fusion consumed at 23° C. is the amount of energy absorbed by the sample to change from the solid state to the state that it has at 23° C., comprising a liquid fraction and a solid fraction.

The liquid fraction of the pasty compound, measured at 32° C., for example, may be present in an amount ranging from 30% to 100% by weight of the compound, further, for example, from 80% to 100% and even further, for example, from 90% to 100% by weight of the compound. When the liquid fraction of the pasty compound measured at 32° C. is equal to 100%, the temperature of the end of the melting range of the pasty compound is less than or equal to 32° C.

The liquid fraction of the pasty compound measured at 32° C. is equal to the ratio of the heat of fusion consumed at 32° C. to the heat of fusion of the pasty compound. The heat of fusion consumed at 32° C. is calculated in the same manner as the heat of fusion consumed at 23° C.

The pasty compound may be chosen from synthetic compounds and compounds of plant origin. A pasty compound may be obtained by synthesis from starting materials of plant origin.

The pasty compound may be, for example, chosen from:
polymer or non-polymer silicone compounds,
polymer or non-polymer fluoro compounds,
vinyl polymers, for example:
   olefin homopolymers
   olefin copolymers
   hydrogenated diene homopolymers and copolymers
   linear or branched oligomers, which are homopolymers or copolymers of alkyl (meth)acrylates, for example, comprising a C₈-C₃₀ alkyl group
   oligomers, which are homopolymers and copolymers of vinyl esters comprising C₈-C₃₀ alkyl groups
   oligomers, which are homopolymers and copolymers of vinyl ethers comprising C₈-C₃₀ alkyl groups,
liposoluble polyethers resulting from the polyetherification between at least one C₂-C₁₀₀, for example, C₂-C₅₀ diols,
esters,
and mixtures thereof.

The pasty compound may be a polymer, for example, a hydrocarbon-based polymer.

Silicone and/or Fluoro Pasty Compounds

A silicone and fluoro pasty compound which may be used is polymethyl trifluoropropyl methylalkyl dimethylsiloxane, sold under the name X22-1088 by Shin-Etsu.

Polyether Pasty Compounds

Among the liposoluble polyethers that may be used, for example, are copolymers of ethylene oxide and/or of propylene oxide with C₆-C₃₀ long-chain alkylene oxides, for example, wherein the weight ratio of the ethylene oxide and/or of the propylene oxide to the alkylene oxides in the copolymer is from 5:95 to 70:30. In this family, non-limiting mention can be made of copolymers wherein the long-chain alkylene oxides are arranged in blocks with an average molecular weight of from 1000 to 10,000, for example, a polyoxyethylene/polydodecyl glycol block copolymer such as the ethers of dodecanediol (22 mol) and of polyethylene glycol (45 EO) sold under the brand name Elfacos ST9 by Akzo Nobel.

Among the esters that may be used, for example, are:
esters of a glycerol oligomer, for example, diglycerol esters, for example, condensates of adipic acid and of glycerol, for which some of the hydroxyl groups of the glycerols have reacted with a mixture of fatty acids such as stearic acid, capric acid, stearic acid and isostearic acid and 12-hydroxystearic acid, for instance those sold under the brand name Softisan 649 by the company Sasol,
arachidyl propionate sold under the brand name Waxenol 801 by Akzo,
phytosterol esters,
non-crosslinked polyesters resulting from polycondensation between a linear or branched C₄-C₅₀ dicarboxylic acid or polycarboxylic acid and a C₂-C₅₀ diol or polyol,
aliphatic esters of an ester resulting from the esterification of an aliphatic hydroxycarboxylic acid ester with an aliphatic carboxylic acid; and mixtures thereof.

The aliphatic carboxylic acid may comprise from 4 to 30, for example, from 8 to 30 carbon atoms. It may be, for example, chosen from hexanoic acid, heptanoic acid, octanoic acid, 2-ethylhexanoic acid, nonanoic acid, decanoic acid, undecanoic acid, dodecanoic acid, tridecanoic acid, tetradecanoic acid, pentadecanoic acid, hexadecanoic acid, hexyldecanoic acid, heptadecanoic acid, octadecanoic acid, isostearic acid, nonadecanoic acid, eicosanoic acid, isoarachidic acid, octyldodecanoic acid, heneicosanoic acid and docosanoic acid, and mixtures thereof.

The aliphatic carboxylic acid may be, for example, branched.

The aliphatic hydroxycarboxylic acid ester may be, for example, derived from a hydroxylated aliphatic carboxylic acid comprising from 2 to 40 carbon atoms, for example, from 10 to 34 carbon atoms and further, for example, from 12 to 28 carbon atoms, and from 1 to 20 hydroxyl groups, for example, from 1 to 10 hydroxyl groups and further, for example, from 1 to 6 hydroxyl groups. The aliphatic hydroxycarboxylic acid ester may be chosen from:
a) partial or total esters of saturated linear monohydroxylated aliphatic monocarboxylic acids;
b) partial or total esters of unsaturated monohydroxylated aliphatic monocarboxylic acids;
c) partial or total esters of saturated monohydroxylated aliphatic polycarboxylic acids;
d) partial or total esters of saturated polyhydroxylated aliphatic polycarboxylic acids;
e) partial or total esters of C₂ to C₁₆ aliphatic polyols that have reacted with a monohydroxylated or polyhydroxylated aliphatic monocarboxylic or polycarboxylic acid,
and mixtures thereof.

The aliphatic esters of an ester may be chosen from:
the ester resulting from the esterification reaction of hydrogenated castor oil with isostearic acid in proportions of 1 to 1 (1/1) or hydrogenated castor oil monoisostearate,
the ester resulting from the esterification reaction of hydrogenated castor oil with isostearic acid in proportions of 1 to 2 (1/2) or hydrogenated castor oil diisostearate,
the ester resulting from the esterification reaction of hydrogenated castor oil with isotearic acid in proportions of 1 to 3 (1/3) or hydrogenated castor oil triisostearate,
and mixtures thereof.

Among the pasty compounds of plant origin which may be chosen is an oxyethylenated (5 EO) oxypropylenated (5 PO) mixture of soybean sterols and of pentaerythritol, sold under the reference Lanolide by the company Vevy.

A pasty fatty substance that may be used is a block copolymer of ethylene oxide and/or propylene oxide and of an alkylene oxide comprising from 6 to 40 carbon atoms, the copolymer having a weight-average molecular weight ranging from 5000 to 8000.

For example, the alkylene oxide of the copolymer may comprise from 6 to 30 carbon atoms, for example, from 8 to 20 carbon atoms, further, for example, from 10 to 18 carbon atoms and even further, for example, from 10 to 14 carbon atoms.

The weight-average molecular weight of the copolymer ranges from 5000 to 8000, for example, from 5500 to 7000, further, for example, from 5500 to 6500 and even further, for example, from 5800 to 6200.

The copolymer may comprise from 35 to 55 ethylene oxide and/or propylene oxide units and from 15 to 30 alkylene oxide units comprising from 6 to 40 carbon atoms.

For example, the copolymer is such that the ratio between the number of ethylene oxide and/or propylene oxide units and the number of alkylene oxide units comprising from 6 to 40 carbon atoms can range from 1.5 to 2.5, for example, from 1.8 to 2.3 and further, for example, from 1.9 to 2.1.

Such copolymers are described, for example, in French Patent Application No. FR-A-2 425 848 and sold under the name “Elfacos® ST 9” by the company Akzo Nobel.

The pasty fatty substance may be present in the composition according to the disclosure in an amount ranging from 1% to 25% by weight, for example, ranging from 1% to 18% by weight and further, for example, ranging from 1% to 10% by weight relative to the total weight of the composition.

The solid emulsion according to the disclosure has a hardness after at least one month at 37° C. wherein the penetration force is greater than 45 g, for example, ranging from 45 g to 150 g.

As used herein, the term “hardness after at least one month at 37° C.” means the hardness measured after storing the solid emulsion for at least one month at 37° C., for example, one month starting from the end of preparation of the solid emulsion.

For example, the solid emulsion has a hardness wherein the penetration force after 24 hours at 20° C. (for example, starting from the end of preparation of the solid emulsion) and after at least one month at 37° C. is greater than or equal to 45 grams (g), for example, ranging from 45 g to 150 g.

For example, the hardness of the solid emulsion ranges from 50 g to 130 g, for example, from 60 g to 130 g and even further, for example, from 70 g to 130 g.

The hardness of the emulsion is measured according to the following protocol:

At the end of preparation of the emulsion, it is poured while hot into a dish and is maintained at 20° C. for 24 hours. The penetration force of the solid emulsion is then measured using a texturometer sold under the name TA-XT2i by the company Rheo, equipped with a TA24 measuring spindle 4 mm in diameter, from the company Stevens, using the following measuring conditions:
trigger force=1.0 g
pre-speed=2.0 mm/s
penetration speed=0.5 mm/s
penetration depth=2 mm

The penetration force expressed in grams is read on the machine.

The solid emulsion is then stored for one month at 37° C., and the emulsion is then placed at 20° C. for 24 hours before measuring the hardness (termed the hardness after one month at 37° C.) under the same conditions described above.

The emulsion according to the disclosure may comprise at least one filler.

As used herein, the term “fillers” mean colorless or white, mineral or synthetic particles of any shape, which are insoluble in the medium of the composition irrespective of the temperature at which the composition is manufactured.

The fillers may be mineral or organic and of any shape, in platelet form, spherical or oblong, irrespective of the crystallographic shape (for example lamellar, cubic, hexagonal, orthorhombic, etc.). Non-limiting mention may be made of talc, mica, silica, kaolin, powders of polyamide (Nylon®), of poly-β-alanine and of polyethylene, powders of tetrafluoroethylene polymers (Teflon®), lauroyllysine, starch, boron nitride, hollow polymer microspheres such as those of polyvinylidene chloride/acrylonitrile, for instance Expancel® (Nobel Industrie), acrylic acid copolymers, silicone resin microbeads (for example Tospearls® from Toshiba), polyorganosiloxane elastomer particles, precipitated calcium carbonate, magnesium carbonate and magnesium hydrocarbonate, hydroxyapatite, hollow silica microspheres, glass or ceramic microcapsules, and metal soaps derived from organic carboxylic acids comprising from 8 to 22 carbon atoms, for example, from 12 to 18 carbon atoms, for example zinc stearate, magnesium stearate, lithium stearate, zinc laurate or magnesium myristate.

The fillers may be present in the composition in an amount ranging from 0.1% to 25% by weight, for example, ranging from 1% to 20% by weight and further, for example, ranging from 5% to 15% by weight relative to the total weight of the composition.

According to one embodiment of the disclosure, the emulsion may comprise polymethyl methacrylate particles. For example, these particles are not film-forming, i.e. they do not form a continuous film when they are placed onto a support such as the skin.

The polymethyl methacrylate powders are generally in the form of hollow or solid white spherical particles generally with a number-average size of micrometer order, for example, ranging from 3 to 15 microns and further, for example, ranging from 3 to 10 microns. The expression “number-average size” denotes the size given by the statistical particle size distribution to half of the population, referred to as D50.

It is also possible to characterize these polymethyl methacrylate particles by their density, which can vary, for example, as a function of the size of the spherical cavity of the said particles.

In the context of the present disclosure, this density is assessed according to the following protocol, referred to as the packed density:
m=40 g of powder is poured into a measuring cylinder; the measuring cylinder is then placed on a Stav 2003 machine from Stampf Volumeter; the measuring cylinder is then subjected to 1500 packing motions; the final volume Vf of packed powder is then measured directly on the measuring cylinder. The packed density is determined by the ratio m/Vf, in this instance 40/Vf (Vf expressed in cm³ and m in g).

For example, the density of the polymethyl methacrylate particles that may be used according to the disclosure may range from 0.3 to 1.5, for example, from 0.5 to 1.5 and further, for example, from 1 to 1.5.

As non-limiting illustrations of the polymethyl methacrylates that are suitable for the disclosure, mention may be made of the polymethyl methacrylate particles sold by the company Matsumoto Yushi Co. under the name “Micropearl M100”, by the company LCW under the name “Covabead LH 85” and those sold by the company Nihon Junyaku under the name “Jurymer MB1”.

The polymethyl methacrylate particles may be present in an amount ranging from 0.5% to 10% by weight, for example, ranging from 0.5% to 7% by weight, further for example, ranging from 1% to 20% by weight and even further, for example, ranging from 0.5% to 5% by weight, relative to the total weight of the composition.

The emulsion according to the disclosure may comprise at least one colorant that may be chosen from water-soluble or liposoluble dyes, pigments and nacres, and mixtures thereof.

As used herein, the term “pigments” means white or colored, mineral or organic particles, which are insoluble in the liquid organic phase, and which are intended to color and/or opacify the composition.

As used herein, the term “nacres” means iridescent particles, produced, for example, by certain molluscs in their shell or, alternatively, synthesized, which are insoluble in the medium of the composition.

As used herein, the term “dyes” means compounds, generally organic compounds, which are soluble in fatty substances, for instance oils, or in an aqueous-alcoholic phase.

The pigments may be mineral or organic pigments. For example, pigments that may be used include metal oxides, for instance iron oxides (for example, yellow, red, brown and black iron oxide), titanium dioxides, cerium oxide, zirconium oxide and chromium oxide; manganese violet, ultramarine blue, Prussian blue, cobalt blue, ferric blue, bismuth oxychloride, nacre, mica coated with titanium or with bismuth oxychloride, colored nacreous pigments such as titanium mica with iron oxides, titanium mica with, for example, ferric blue or chromium oxide, titanium mica with an organic pigment of the abovementioned type and nacreous pigments based on bismuth oxychloride, and mixtures thereof.

Iron oxide or titanium oxide pigments may be used.

The pigments may be treated with a hydrophobic agent to make them compatible with the organic phase of the composition. The hydrophobic-treatment agent may be chosen from silicones, for instance methicones, dimethicones or perfluoroalkyl-silanes; fatty acids, for instance stearic acid; metal soaps, for instance aluminium dimyristate, the aluminium salt of hydrogenated tallow glutamate, perfluoroalkyl phosphates, perfluoroalkylsilanes, perfluoroalkylsilazanes, polyhexafluoropropylene oxides, polyorganosiloxanes comprising perfluoroalkyl perfluoropolyether groups, and amino acids; N-acylamino acids or salts thereof; lecithin, isopropyl triisostearyl titanate, and mixtures thereof.

The N-acylamino acids may comprise an acyl group comprising from 8 to 22 carbon atoms, for instance a 2-ethylhexanoyl, caproyl, lauroyl, myristoyl, palmitoyl, stearoyl or cocoyl group. The salts of these compounds may be aluminium, magnesium, calcium, zirconium, zinc, sodium or potassium salts. The amino acid may be, for example, lysine, glutamic acid or alanine.

As used herein, the term “alkyl” mentioned in the compounds mentioned above may be chosen from an alkyl group comprising from 1 to 30 carbon atoms, for example, comprising from 5 to 16 carbon atoms.

Hydrophobic-treated pigments are described, for example, in European Patent Application No. EP-A-1 086 683.

The liposoluble dyes are, for example, Sudan Red, D&C Red No. 17, D&C Green No. 6, β-carotene, soybean oil, Sudan Brown, D&C Yellow No. 11, D&C Violet No. 2, D&C Orange No. 5, quinoline yellow, annatto and bromo acids.

The water-soluble dyes are, for example, beetroot juice, methylene blue and caramel.

The dyestuffs may be present in an amount ranging from 0.5% to 30% by weight, for example, ranging from 3% to 20% by weight and further, for example, ranging from 5% to 15% by weight, relative to the total weight of the composition.

According to one embodiment of the disclosure, the emulsion may comprise at least one hydrophobic coated pulverulent material, for example, in an amount of less than or equal to 20% by weight relative to the total weight of the composition. The said pulverulent material may be chosen from pulverulent fillers and dyestuffs, for instance the fillers and pigments as described above. The said pulverulent material is coated with a hydrophobic agent as described above.

The composition may comprise at least one other common cosmetic ingredient that may be chosen, for example, from hydrophilic or lipophilic gelling agents and/or thickeners, antioxidants, fragrances, preserving agents, neutralizers, sunscreens, vitamins, moisturizers, self-tanning compounds, anti-wrinkle active agents, emollients, hydrophilic or lipophilic active agents, free-radical scavengers, sequestering agents and film-forming agents.

Needless to say, a person skilled in the art will take care to select this or these optional compound(s), and/or the amount thereof, wherein the advantageous properties of the composition according to the disclosure are not, or are not substantially, adversely affected by the envisaged addition.

The composition according to the disclosure may be, for example, prepared according to the following procedure:

The mixture of the constituents of the oily phase is first prepared by mixing together and heating, at a temperature ranging from 70° C. to 120° C., the waxes, the block copolymer of ethylene oxide and/or of propylene oxide and of an alkylene oxide comprising from 6 to 40 carbon atoms, and the non-volatile oils, followed by adding with stirring, at a temperature ranging from 60° C. to 80° C., the volatile oils, the fillers and the pigments.

The mixture of the constituents of the aqueous phase, comprising the water, the surfactants and the water-miscible solvents is prepared separately, with heating to a temperature ranging from 60° C. to 80° C.

Next, the aqueous phase is added to the oily phase, at a temperature ranging from 60 to 80° C. and the resulting mixture is stirred using a turbomixer until the water-in-oil emulsion is obtained. The emulsion is then poured into a container, for example a dish, and then cooled to room temperature until the solid emulsion is obtained.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the embodiments disclosed herein. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosed embodiments are approximations, unless otherwise indicated the numerical values set forth in the specific examples are reported as precisely as possible. Any numerical value, however, inherently contain certain errors necessarily resulting from the standard deviation found in their respective testing measurements.

The embodiments disclosed herein are illustrated in greater detail by the examples described below.

### EXAMPLE 1

A solid foundation having the composition below was prepared:

| Oily phase: | | |
|---|---|---|
| Candelilla wax | 1.8 | g |
| Ozokerite | 1.2 | g |
| Isotridecyl isononanoate | 9.0 | g |
| Cyclopentasiloxane | 22.5 | g |
| Iron oxides coated with disodium stearoyl glutamate and | 2.2 | g |
| aluminium hydroxide | | |
| Titanium dioxide coated with disodium stearoyl glutamate and | 7.8 | g |
| aluminium hydroxide | | |
| Hydrophobic fumed silica (Aerosil R972 from Degussa) | 0.5 | g |
| Silica microbeads | 9.0 | g |
| Polymethyl methacrylate | 3 | g |
| Titanium dioxide nanopigments | 3 | g |
| Copolymer of ethylene oxide (45 EO) and of | 4 | g |
| epoxydodecane (22 mol) sold under the name “Elfacos ® ST | | |
| 9” by the company Akzo Nobel | | |
| Polyglyceryl-4 isostearate | 1.50 | g |
| Aqueous phase: | | |
| Water | 20 | g |
| Butylene glycol | 3 | g |
| Glycerol | 5 | g |
| Magnesium sulphate | 1 | g |
| Cetyl dimethicone copolyol (Abil ® EM90 from the company | 2 | g |
| Goldschmidt) | | |
| Mixture of oxyethylenated oxypropylenated (18 EO/18 PO) | 2 | g |
| polydimethylsiloxane, cyclopentasiloxane and water (10/88/2) | | |
| (DC 2-5225 C from Dow Corning) | | |
| Preserving agents | 1.50 | g |

The composition was prepared according to the following procedure:

The mixture comprising the waxes, the copolymer Elfacos ST 9 and the non-volatile oils was heated to 90° C. until a clear, uniform mixture was obtained; the volatile oils, the pigments and the fillers were then added with stirring, at 70° C. The mixture of the ingredients of the aqueous phase was then prepared with heating to 70° C., and the aqueous phase was then introduced at 70° C. into the oily phase, with stirring until the water-in-oil emulsion was obtained.

The emulsion was poured at 70° C. into a preheated dish and was then allowed to cool to room temperature until the solid emulsion was obtained.

A solid foundation was obtained, having a hardness of 98 g measured at 24 hours at 20° C. and a hardness of 87 g measured after storage at 37° C. for one month, the hardness measurements were performed according to the protocol described above. This solid foundation thus had good hardness stability after storage for one month.

When applied to the skin, the foundation was rich and creamy.

### EXAMPLE 2

A solid foundation having the composition below was prepared:

| Oily phase: | | |
|---|---|---|
| Candelilla wax | 1.8 | g |
| Ozokerite | 1.2 | g |
| Isodecyl neopentanoate | 10.2 | g |
| Cyclopentasiloxane | 22.5 | g |
| Iron oxides coated with disodium stearoyl glutamate and | 2.4 | g |
| aluminium hydroxide | | |
| Titanium dioxide coated with disodium stearoyl glutamate and | 7.6 | g |
| aluminium hydroxide | | |
| Hydrophobic fumed silica (Aerosil R972 from Degussa) | 0.5 | g |
| Silica microbeads | 9.0 | g |
| Polymethyl methacrylate | 3 | g |
| Titanium dioxide nanopigments | 3 | g |
| Copolymer of ethylene oxide (45 EO) and of | 4 | g |
| epoxydodecane (22 mol) sold under the name “Elfacos ® ST | | |
| 9” by the company Akzo Nobel | | |
| Polyglyceryl-3 diisostearate | 0.3 | g |
| Aqueous phase: | | |
| Water | 20 | g |
| Butylene glycol | 3 | g |
| Glycerol | 5 | g |
| Magnesium sulfate | 1 | g |
| Cetyl dimethicone copolyol (Abil ® EM 90 from the | 2 | g |
| company Goldschmidt) | | |
| Mixture of oxyethylenated oxypropylenated (18 EO/18 PO) | 2 | g |
| polydimethylsiloxane, cyclopentasiloxane and water (10/88/2) | | |
| (DC 2-5225 C from Dow Corning) | | |
| Preserving agents | 1.50 | g |

The emulsion was prepared in the same manner as that of Example 1.

A solid foundation was obtained, having a hardness of 87 g measured at 24 hours at 20° C. and a hardness of 78 g measured after storage at 37° C. for one month, the hardness measurements were performed according to the protocol described above. This solid foundation thus had good hardness stability after storage for one month.

When applied to the skin, the foundation was rich and creamy.

### EXAMPLE 3

A solid foundation having the composition below was prepared:

| Oily phase: | | |
|---|---|---|
| Candelilla wax | 1.8 | g |
| Ozokerite | 1.2 | g |
| Isotridecyl isononanoate | 9.0 | g |
| Cyclopentasiloxane | 22.5 | g |
| Iron oxides coated with disodium stearoyl glutamate and | 2.4 | g |
| aluminium hydroxide | | |
| Titanium dioxide coated with disodium stearoyl glutamate and | 7.8 | g |
| aluminium hydroxide | | |
| Hydrophobic fumed silica (Aerosil R972 from Degussa) | 0.5 | g |
| Silica microbeads | 9.0 | g |
| Polymethyl methacrylate | 3 | g |
| Titanium dioxide nanopigments | 3 | g |
| Copolymer of ethylene oxide (45 EO) and of | 4 | g |
| epoxydodecane (22 mol) sold under the name “Elfacos ® ST | | |
| 9” by the company Akzo Nobel | | |
| Polyglyceryl-4 isostearate | 1.50 | g |
| Silicone comprising a polyglycerolated chain (DF6104 by | 2 | g |
| Shin-Etsu) | | |
| Aqueous phase: | | |
| Water | 20 | g |
| Butylene glycol | 3 | g |
| Glycerol | 5 | g |
| Magnesium sulfate | 1 | g |
| Mixture of oxyethylenated oxypropylenated (18 EO/18 PO) | 2 | g |
| polydimethylsiloxane, cyclopentasiloxane and water (10/88/2) | | |
| (DC 2-5225 C from Dow Corning) | | |
| Preserving agents | 1.50 | g |

The emulsion was prepared in the same manner as that of Example 1.

A solid foundation was obtained, having a hardness of 118.2 g measured at 24 hours at 20° C. and a hardness of greater than 45 g measured after storage at 37° C. for one month, the hardness measurements were performed according to the protocol described above.

When applied to the skin, the foundation was rich and creamy.

What is claimed is:

1. A solid water-in-oil emulsion comprising an aqueous phase emulsified with at least one emulsifying surfactant in a fatty phase comprising at least one ester oil and at least one wax, wherein the at least one ester oil is present in an amount greater than the at least one wax, wherein the emulsion has, after at least one month at 37° C., a hardness wherein the penetration force is greater than or equal to 45 g.

2. A solid water-in-oil emulsion comprising an aqueous phase emulsified with an emulsifying surfactant in a fatty phase comprising an ester oil and a wax and at least one compound chosen from
at least two polyols,
at least one pasty fatty substance, and
at least one hydrophobic coated pulverulent material in an amount of less than or equal to 20% by weight, relative to the total weight of the composition,
wherein the emulsion has, after at least one month at 37° C., a hardness wherein the penetration force is greater than or equal to 45 g.

3. An emulsion according to claim 1, comprising at least one compound chosen from
at least two polyols,
at least one pasty fatty substance, and
at least one hydrophobic coated pulverulent material in an amount of less than or equal to 20% by weight, relative to the total weight of the composition.

4. An emulsion according to claim 1, wherein the hardness ranges from 70 g to 130 g.

5. An emulsion according to claim 1, wherein the at least one ester oil is a monoester.

6. An emulsion according to claim 1, wherein the at least one ester oil is a monoester of formula R₁COOR₂
wherein:
R₁ is chosen from linear and branched hydrocarbon-based chains comprising from 4 to 40 carbon atoms, and
R₂ is chosen from branched hydrocarbon-based chains comprising from 3 to 40 carbon atoms.

7. An emulsion according to claim 6, wherein R₁ is chosen from linear and branched hydrocarbon-based chains comprising from 7 to 20 carbon atoms.

8. An emulsion according to claim 6, wherein R₂ is chosen from branched hydrocarbon-based chains comprising from 16 to 26 carbon atoms.

9. An emulsion according to claim 1, wherein the at least one ester oil is chosen from isodecyl neopentanoate, isocetyl octanoate, isononyl isononanoate, isodecyl isononanoate, tridecyl isononanoate, hexyl laurate, 2-hexyldecyl laurate, isopropyl myristate, isocetyl myristate, isotridecyl myristate, 2-octyldodecyl myristate, isopropyl palmitate, 2-ethylhexyl palmitate, isooctyl palmitate, isocetyl palmitate, isodecyl palmitate, isostearyl palmitate, 2-octyldecyl palmitate, isopropyl isostearate, 2-octyldodecyl stearate, isostearyl isostearate and 2-octyldodecyl erucate.

10. An emulsion according to claim 1, wherein the at least one ester oil is present in an amount ranging from 5% to 20% by weight, relative to the total weight of the composition.

11. An emulsion according to claim 10, wherein the at least one ester oil is present in an amount ranging from 8% to 12% by weight, relative to the total weight of the composition.

12. An emulsion according to claim 1, wherein the at least one wax has a hardness ranging from 5 MPa to 9 MPa.

13. An emulsion according to claim 1, wherein the at least one wax has a hardness ranging from 7 MPa to 9 MPa.

14. An emulsion according to claim 1, wherein the at least one wax is chosen from carnauba wax, microcrystalline waxes, ozokerites, hydrogenated jojoba oil, polyethylene waxes, poly(C₂₄-C₂₈)alkyl methyl dimethyl siloxane waxes, palm butter, C₂₀-C₄₀ alkyl stearate, stearyl benzoate and shellac wax, and mixtures thereof.

15. An emulsion according to claim 1, wherein the at least one wax is chosen from carnauba wax, ozokerites, hydrogenated jojoba oil and polyethylene waxes.

16. An emulsion according to claim 1, wherein the at least one wax is chosen from candelilla wax and ozokerite, and mixtures thereof.

17. An emulsion according to claim 1, wherein the at least one wax is present in an amount ranging from 1% to 10% by weight, relative to the total weight of the composition.

18. An emulsion according to claim 17, wherein the at least one wax is present in an amount ranging from 2% to 5% by weight, relative to the total weight of the composition.

19. An emulsion according to claim 1, wherein the at least one emulsifying surfactant is chosen from fatty acid esters of polyols; alkyl or alkoxy dimethicone copolyols comprising an alkyl or alkoxy chain that is pendent or at the end of the silicone skeleton; polyoxyalkylenated fatty acid esters of glycol; polyhydroxylated silicones; emulsifying silicone elastomers, and mixtures thereof.

20. An emulsion according to claim 19, wherein the fatty acid esters of polyols are chosen from glyceryl or sorbitol mono-, di-, tri- or sesqui-oleates or -stearates, and glyceryl or polyethylene glycol laureates.

21. An emulsion according to claim 19, wherein the polyhydroxylated silicones are chosen from polyglycerolated silicones.

22. An emulsion according to claim 19, wherein the alkyl dimethicone copolyols are chosen from: 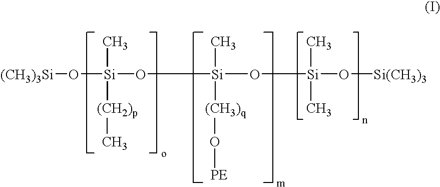
wherein:
PE is (—C₂H₄O)ₓ—(C₃H₆O)_{y}—R,
wherein:
R is chosen from a hydrogen atom and alkyl radicals comprising from 1 to 4 carbon atoms,
x is a number ranging from 0 to 100, and
y is a number ranging from 0 to 80, wherein x and y are not simultaneously equal to 0,
m is a number ranging from 1 to 40,
n is a number ranging from 10 to 200,
o is a number ranging from 1 to 100,
p is a number ranging from 7 to 21, and
q is a number ranging from 0 to 4.

23. An emulsion according to claim 1, wherein the at least one emulsifying surfactant is present in an amount ranging from 1% to 10% by weight, relative to the total weight of the composition.

24. An emulsion according to claim 23, wherein the at least one emulsifying surfactant is present in an amount ranging from 2% to 5% by weight, relative to the total weight of the composition.

25. An emulsion according to claim 1, wherein the fatty phase comprises at least one volatile silicone oil.

26. An emulsion according to claim 25, wherein the at least one volatile silicone oil is chosen from octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

27. An emulsion according to claim 25, wherein the at least one volatile silicone oil is present in an amount ranging from 5% to 40% by weight, relative to the total weight of the composition.

28. An emulsion according to claim 25, wherein the at least one volatile silicone oil is present in an amount ranging from 15% to 30% by weight, relative to the total weight of the composition.

29. An emulsion according to claim 25, wherein the water and the at least one volatile silicone oil are present in a total amount of less than or equal to 50% by weight, relative to the total weight of the composition.

30. An emulsion according to claim 29, wherein the water and the at least one volatile silicone oil are present in a total amount ranging from 40% to 50% by weight, relative to the total weight of the composition.

31. An emulsion according to claim 1, comprising at least one additional oil other than the at least one ester oil and at least one volatile silicone oil.

32. An emulsion according to claim 31, wherein the at least one additional oil is chosen from volatile or non-volatile hydrocarbon-based oils and fluoro oils.

33. An emulsion according to claim 31, wherein the at least one additional oil is present in an amount ranging from 0.1% to 10% by weight, relative to the total weight of the composition.

34. An emulsion according to claim 33, wherein the at least one additional oil is present in an amount ranging from 0.1% to 5% by weight, relative to the total weight of the composition.

35. An emulsion according to claim 1, wherein the water is present in an amount ranging from 10% to 40% by weight, relative to the total weight of the composition.

36. An emulsion according to claim 1, wherein the water is present in an amount ranging from 15% to 25% by weight, relative to the total weight of the composition.

37. An emulsion according to claim 1, comprising at least one water-miscible solvent chosen from monoalcohols comprising from 2 to 6 carbon atoms, polyols comprising from 2 to 20 carbon atoms and glycol ethers comprising from 3 to 16 carbon atoms, and mixtures thereof.

38. An emulsion according to claim 37, wherein the at least one water-miscible solvent is chosen from glycerol, propylene glycol, butylene glycol, pentylene glycol, hexylene glycol, dipropylene glycol, diethylene glycol, mono-, di- or tripropylene glycol (C₁-C₄)alkyl ethers, mono-, di- or triethylene glycol (C₁-C₄)alkyl ethers, ethanol and isopropanol, and mixtures thereof.

39. An emulsion according to claim 37, wherein the at least one water-miscible organic solvent is present in an amount ranging from 1% to 20% by weight, relative to the total weight of the composition.

40. An emulsion according to claim 39, wherein the at least one water-miscible organic solvent is present in an amount ranging from 3% to 15% by weight, relative to the total weight of the composition.

41. An emulsion according to claim 3, comprising at least one water-miscible polyol having 3 carbon atoms and at least one water-miscible polyol having more than 3 carbon atoms.

42. An emulsion according to claim 41, wherein the at least one water-miscible polyol having more than 3 carbon atoms comprises from 4 to 6 carbon atoms.

43. An emusion according to claim 41, wherein the at least one water-miscible polyol having more than 3 carbon atoms is chosen from butylene glycol, pentylene glycol, hexylene glycol, dipropylene glycol, diethylene glycol, and mixtures thereof.

44. An emulsion according to claim 3, wherein the at least one pasty fatty substance is chosen from a block copolymer of ethylene oxide and/or propylene oxide and of an alkylene oxide comprising from 6 to 40 carbon atoms, the copolymer having a weight-average molecular weight ranging from 5000 to 8000.

45. An emulsion according to claim 1, comprising at least one filler.

46. An emulsion according claim 45, wherein the filler is chosen from talc, mica, silica, kaolin, polyamide powders, poly-β-alanine powders, polyethylene powders, tetrafluoroethylene polymer powders, lauroyllysine, starch, boron nitride, hollow microspheres of polyvinylidene chloride/acrylonitrile, hollow microspheres of acrylic acid copolymers, silicone resin microbeads, polyorganosiloxane elastomer particles, precipitated calcium carbonate, magnesium carbonate and magnesium hydrocarbonate, hydroxyapatite, hollow silica microspheres, glass or ceramic microcapsules, and metal soaps derived from organic carboxylic acids comprising from 8 to 22 carbon atoms.

47. An emulsion according to claim 45, wherein the fillers are present in an amount ranging from 0.1% to 25% by weight, relative to the total weight of the composition.

48. An emulsion according to claim 47, wherein the fillers are present in an amount ranging from 5% to 15% by weight, relative to the total weight of the composition.

49. An emulsion according to claim 1, comprising polymethyl methacrylate particles.

50. An emulsion according to claim 49, wherein the polymethyl methacrylate particles are present in an amount ranging from 0.5% to 10% by weight, relative to the total weight of the composition.

51. An emulsion according to claim 50, wherein the polymethyl methacrylate particles are present in an amount ranging from 0.5% to 5% by weight, relative to the total weight of the composition.

52. An emulsion according to claim 1, comprising at least one colorant.

53. An emulsion according to claim 3, wherein the at least one hydrophobic coated pulverulent material is chosen from pulverulent fillers and colorants.

54. An emulsion according to claim 3, wherein the at least one hydrophobic coated pulverulent material is coated with a hydrophobic agent chosen from silicones, fatty acids, metal soaps, perfluoroalkyl phosphates, perfluoroalkyl silanes, perfluoroalkyl silazanes, polyhexafluoropropylene oxides, pqlyorganosiloxanes comprising perfluoroalkyl perfluoropolyether groups, amino acids, N-acylamino acids or salts thereof, lecithin and isopropyl triisostearyl titanate, and mixtures thereof.

55. An emulsion according to claim 1, comprising at least one cosmetic ingredient chosen from hydrophilic or lipophilic gelling agents and/or thickeners, antioxidants, fragrances, preserving agents, neutralizers, sunscreens, vitamins, moisturizers, self-tanning compounds, anti-wrinkle active agents, emollients, hydrophilic or lipophilic active agents, free-radical scavengers, sequestering agents and film-forming agents, and mixtures thereof.

56. A composition comprising a solid water-in-oil emulsion comprising an aqueous phase emulsified with at least one emulsifying surfactant in a fatty phase comprising at least one ester oil and at least one wax, wherein the at least one ester oil is present in an amount greater than the at least one wax and wherein the emulsion has, after at least one month at 37° C., a hardness wherein the penetration force is greater than or equal to 45 g.

57. A composition according to claim 56 wherein the composition is a foundation, an eyeshadow, a makeup rouge, a concealer product, a body makeup product, a lipstick, a skincare base, a care cream; a lipcare composition; an anti-sun composition, a self-tanning composition; a deodorant.

58. A foundation comprising a composition comprising a solid water-in-oil emulsion comprising an aqueous phase emulsified with at least one emulsifying surfactant in a fatty phase comprising at least one ester oil and at least one wax, wherein the at least one ester oil is present in an amount greater than the at least one wax and wherein the emulsion has, after at least one month at 37° C., a hardness wherein the penetration force is greater than or equal to 45 g.

59. Non-therapeutic cosmetic process for making up or caring for the skin, comprising the application to the skin of a composition comprising a solid water-in-oil emulsion comprising an aqueous phase emulsified with at least one emulsifying surfactant in a fatty phase comprising at least one ester oil and at least one wax, wherein the at least one ester oil is present in an amount greater than the at least one wax and wherein the emulsion has, after at least one month at 37° C., a hardness wherein the penetration force is greater than or equal to 45 g.
